(19) United States
(12) Patent Application Publication
(10) Pub. No.: US 2013/0281737 A1
(43) Pub. Date: Oct. 24, 2013

(54) METHOD FOR REDUCING EQUIPMENT FOULING IN (METH)ACRYLIC ACID PRODUCTION PROCESS

(71) Applicants: Marc Charendorff, Houston, TX (US); Jamie Jerrick Juliette, Houston, TX (US); Joy Mendoza, Seabrook, TX (US); Rajesh Shah, Katy, TX (US)

(72) Inventors: Marc Charendorff, Houston, TX (US); Jamie Jerrick Juliette, Houston, TX (US); Joy Mendoza, Seabrook, TX (US); Rajesh Shah, Katy, TX (US)

(73) Assignee: ROHM AND HAAS COMPANY, Philadelphia, PA (US)

(21) Appl. No.: 13/976,278

(22) PCT Filed: Nov. 30, 2011
(86) PCT No.: PCT/US2011/062605

§ 371 Date: Jun. 26, 2013

Related U.S. Application Data
(60) Provisional application No. 61/460,245, filed on Dec. 29, 2010.

(51) Int. Cl. C07C 51/44

(52) U.S. Cl. CPC C07C 51/44
USPC 562/600

(57) ABSTRACT

The present invention provides a method for reducing fouling of equipment during separation and purification steps of (meth)acrylic acid production by early removal of aldehyde impurities by adding a hydrazide compound well upstream of the separation and purification steps. In particular, carbodhydrazide may be added as an aldehyde scavenging agent to aqueous (meth)acrylic acid prior to dehydration and purification steps.

### CROSS-REFERENCE TO RELATED APPLICATION

This application claims priority from provisional application Ser. No. 61/460,245, filed Dec. 29, 2010, which is incorporated herein by reference in its entirety.

### FIELD OF THE INVENTION

The present invention relates to a process for producing (meth)acrylic acid and, more particularly, to a method for reducing fouling of equipment during separation and purification steps of acrylic acid production by removal of aldehyde impurities with a hydrazide compound well upstream of the separation and purification steps.

### BACKGROUND OF THE INVENTION

(Meth)acrylic acid and its esters are industrially important for manufacturing polymers for a very wide range of applications including, but not limited to, adhesives, coatings, films, biomedical carriers and devices, and binders. (Meth)acrylic acid may be produced, among other methods, by catalytic gas-phase oxidation of alkanes, alkanols, alkenes or alkenals containing 3 or 4 carbon atoms. One widely practiced process is, for example, catalytic gas-phase oxidation of propene, acrolein, tert-butanol, iso-butene, iso-butane, iso-butyraldehyde or methacrolein. These starting materials are generally diluted with inert gases such as nitrogen, carbon monoxide, carbon dioxide, saturated hydrocarbons and/or steam, and then contacted with a mixed metal oxide catalyst (for example, containing one or more of molybdenum, vanadium, tungsten and iron), with or without molecular oxygen, at elevated temperatures (e.g., from 200° C. to 400° C.) to be oxidized into (meth)acrylic acid.

Since there are numerous parallel and subsequent reactions occurring in the course of the catalytic vapor phase oxidation, and because of the inert diluent gases used, the resulting mixed gas product contains not only (meth)acrylic acid, but also inert diluent gases, impurities, and byproducts, from which the (meth)acrylic acid has to be separated. Thus, the mixed product gas is next typically subjected to absorption to remove (meth)acrylic acid from some of the byproducts and impurities and form a (meth)acrylic acid solution. It is known to use an absorption solvent such as water or an hydrophobic organic liquid (e.g., without limitation, toluene, methyl isobutyl ketone (MiBK), and diphenyl ether) or the (meth)acrylic acid itself (e.g, as in a fractionating column) for the absorption step. The resulting (meth)acrylic solution is then subjected to further separation and purification steps, such as by azeotropic or simple distillation, or crystallization, or extraction, to produce a crude (meth)acrylic acid product which may or may not be subjected to further purification or reaction as desired, depending on the intended end-use.

Besides byproducts which are comparatively simple to remove from (meth)acrylic acid, such as acetic acid, the mixed gas product also contains aldehyde compounds, which are closely related to (meth)acrylic acid and, therefore, can be difficult to separate from (meth)acrylic acid. The aldehydes present in the oxidation product typically include, for example, one or more of the following: formaldehyde, acetaldehyde, acrolein, methacrolein, propionaldehyde, n-butyraldehyde, benzaldehyde, phthaldehyde, furfural and crotonaldehyde and possibly also maleic anhydride or its acid. The total amount of aldehyde compounds present in the mixed gas product may be up to, or even more than, about 2% by weight based on the total weight of the mixed gas product obtained from the oxidation reaction. Aldehyde compounds, especially the lower molecular C₁ to C₃ analogues (formaldehyde, acetaldehyde, and proprionaldehyde), have been reported to initiate polymerization of (meth)acrylic acid in separations equipment such as distillation columns, reboilers and heat exchanger equipment. In particular, formaldehyde has been shown in the art as contributing to solids when placed in contact with common polymerization inhibitors such as phenothiazine (PTZ), hydroquinone (HQ), and hydroquinone monomethyl ether (MeHQ) (see, U.S. Patent Application Publication No. US2007/0167650). Furfural (C₅) and acrolein have also been reported as fouling contributors in the processing of (meth)acrylic acid. U.S. Patent Application Publication No. US2001/0004960 teaches addition of hydrazine as an aldehyde scavenger in crude (meth)acrylic acid for removal of furfural and acrolein. U.S. Patent Application Publication No. US2005/0187495 describes the use of hydrazine, hydrazine hydrates and mixtures thereof for removal of aldehydes and maleic compounds from crude acrylic acid after separation and purification by azeotropic distillation using a heavy solvent such as MiBK, toluene, and the like. U.S. Pat. No. 5,961,790 teaches removal of aldehydes from (meth)acrylic acid by addition of hydrazides to crude acrylic acid

U.S. Pat. No. 6,179,966 discloses the addition of primary and secondary amines, hydrazines, and related derivatives and salts to aqueous acrylic acid, prior to “evaporation,” which is essentially vaporization, of the aqueous acrylic acid prior to its being subjected to the usual azeotropic distillation separations to produce crude acrylic acid.

U.S. Patent Application Publication No. US2001/0016668 describes a process for producing (meth)acrylic acid involving absorption of (meth)acrylic acid from a mixed product gas, followed by formation of crude (meth)acrylic acid by solvent extraction or azeotropic distillation. In this process, an aldehyde treating compound is added to the crude (meth)acrylic acid, which is then subjected to vacuum distillation to obtain high purity (meth)acrylic acid and the waste liquid generated by the vacuum distillation is returned to the absorbing or separating steps. The aldehyde treating agent is a primary amine and/or a salt thereof which may be a hydrazine hydrate or a phenyl hydrazine, among other specified amines.

U.S. Pat. No. 7,393,976 teaches addition of an aldehyde treating compound which may be, among others, sulfuric acid, hydrazine compounds, amine compounds, and hydrazide compounds, to one or more distillation columns, after absorption and water removal steps to produce concentrated aqueous (meth)acrylic acid.

Similarly, U.S. Pat. No. 5,482,597 describes addition of hydrazine or dihydrazine of a C₄-C₈ dicarboxylic acid to one or more distillation columns, after absorption using a non-aqueous heavy solvent to produce a (meth)acrylic acid solution which is subjected to purification by distillation. U.S. Pat. Nos. 5,961,790 and 6,228,227 both teach addition of a primary amine or a salt thereof, such as a hydrazide of an organic carboxylic acid, to one or more distillation columns, in which a (meth)acrylic acid solution comprising an inert hydrophobic organic liquid solvent is subjected to purification by distillation.

The present invention provides a more effective and efficient method for reducing downstream fouling of separation equipment in a process for producing (meth)acrylic acid by removing aldehydes, such as formaldehyde, by adding a hydrazide compound, such as carbohydrazide, upstream of the water removal and distillation steps of the process.

### SUMMARY OF THE INVENTION

The present invention provides a method for reducing fouling of equipment during purification of (meth)acrylic acid in a process which involves the steps of:
A) producing a mixed product gas comprising (meth)acrylic acid, one or more aldehyde compounds, one or more light end compounds each having a lighter boiling point than (meth)acrylic acid, and one or more heavy end compounds each having a higher boiling point than (meth)acrylic acid;
B) producing aqueous (meth)acrylic acid from the mixed product gas comprising the (meth)acrylic acid, the one or more aldehyde compounds, the one or more light end compounds, the one or more heavy end compounds, and water;
C) removing at least a portion of the water from the aqueous (meth)acrylic acid to produce a concentrated aqueous (meth)acrylic acid;
D) purifying the concentrated aqueous (meth)acrylic acid by removing at least a portion of the one or more heavy end components; and
E) optionally, purifying the concentrated aqueous (meth)acrylic acid by removing an additional portion of the one or more light end components.

More particularly, the method of the present invention comprises removing at least a portion of the one or more aldehyde compounds from the aqueous (meth)acrylic acid by adding at least one hydrazide compound either 1) during step B) of producing the aqueous (meth)acrylic acid; or 2) after step B), to the aqueous (meth)acrylic acid, and prior to any of the water removing and purifying steps C), D) and E); or 3) both 1) and 2).

The hydrazide compound has the following formula:

H₂N—NHR₁

wherein R₁ is C(O)NH₂) or C(O)NHNH₂.

In some embodiments, the hydrazide compound is semicarbohydrazide. In other embodiments the hydrazide compound is carbohydrazide.

The hydrazide compound is added in an amount of from 0.5 to 5 moles per 1 mole of aldehyde compound present in the aqueous (meth)acrylic acid.

The step producing aqueous (meth)acrylic acid from the mixed product gas may be accomplished by subjecting the mixed product gas to absorption with a solvent comprising water to remove at least a portion of the one or more light end compounds.

### DETAILED DESCRIPTION OF THE INVENTION

As used herein, the term “(meth)acrylic acid” means acrylic acid or methacrylic acid.

Processes for the production of (meth)acrylic acid are, in general, well understood and practiced by persons of ordinary skill in the relevant art and tend to involve a similar sequence of process steps including production of a mixed gas product which comprises (meth)acrylic acid, capturing (meth)acrylic acid in a solution, and subjecting the (meth)acrylic acid solution to one or more further purification steps. The method of the present invention is advantageously applicable to production processes wherein the (meth)acrylic acid is captured by absorption to form an aqueous (meth)acrylic acid, which is then subjected to a water removal step prior to further separation and purification steps.

More particularly, the present invention provides a method for reducing fouling of equipment during purification of (meth)acrylic acid in a process for producing (meth)acrylic acid which generally involves a first step of producing a mixed product gas comprising (meth)acrylic acid, one or more aldehyde compounds, one or more light end compounds each having a lower boiling point than (meth)acrylic acid, and one or more heavy end compounds each having a higher boiling point than (meth)acrylic acid. While the method of producing the mixed product gas comprising (meth)acrylic acid is not particularly critical or limited, one method would be catalytic vapor phase oxidation of alkanes, alkanols, alkenes or alkenals containing 3 or 4 carbon atoms, such as propane, propene, acrolein, tert-butanol, iso-butene, iso-butane, iso-butyraldehyde or methacrolein. The starting materials for the oxidation reaction may be diluted with inert gases such as nitrogen, carbon monoxide, carbon dioxide, saturated hydrocarbons and/or steam, and then contacted with a mixed metal oxide catalyst (for example, containing one or more of molybdenum, vanadium, tungsten and iron), with or without molecular oxygen, at elevated temperatures (e.g., from 200° C. to 400° C.).

Aqueous (meth)acrylic acid is then recovered from the mixed product gas, such as by subjecting the mixed product gas to absorption using a solvent comprising water or the (meth)acrylic acid as would be common in a fractionating column. During absorption, at least a portion of the one or more light end compounds are separated from the mixed product gas. As expected, the resulting aqueous (meth)acrylic acid comprises (meth)acrylic acid, one or more aldehyde compounds, one or more light end compounds, one or more heavy end compounds, and water. Then, at least a portion of the water is removed from the aqueous (meth)acrylic acid to produce a concentrated aqueous (meth)acrylic acid, in preparation for separation steps more particularly designed to remove light and heavy end compounds from the (meth)acrylic acid. As known by persons of ordinary skill in the relevant art, water may be removed from the aqueous (meth)acrylic acid by any conventional method, such as, but not limited to, rectification, distillation, extraction, or crystallization.

In order to reduce formation of polymer solids which cause fouling of downstream separation equipment, at least a portion of aldehyde compounds such as, without limitation, formaldehyde, are removed from the aqueous (meth)acrylic acid by adding a hydrazide compound to the aqueous (meth)acrylic acid prior to the water removal step and prior to any further separation and purification steps.

In some embodiments, in accordance with the method of the present invention, the hydrazide compound may be added to the aqueous (meth)acrylic acid after its formation (e.g., by absorption). In some embodiments, the hydrazide compound may be added to the absorption step, i.e., during production of the aqueous (meth)acrylic acid (e.g., by absorption). In still other embodiments, in accordance with the present invention, the hydrazide compound may be added to both the absorption step, as well as to the aqueous (meth)acrylic acid after its formation by absorption, and prior to removing water to produce the concentrated aqueous (meth)acrylic acid.

The hydrazide compound has the following formula:

H₂N—NHR₁

wherein R₁ is C(O)NH₂) or C(O)NHNH₂. The hydrazide compound is selected from the group consisting of: semicarbohydrazide, carbohydrazide, and mixtures thereof. In one embodiment, the hydrazide compound is carbohydrazide. The hydrazide compound may suitably be added in an amount of from 0.5 to 5 moles per 1 mole of aldehyde compound present in the aqueous (meth)acrylic acid. For example, the amount of hydrazide compound added may be from 0.5 to 2 moles, or even from 0.5 to 1 mole, per 1 mole of aldehyde compound.

In contrast to amine based aldehyde scavengers, including hydrazine, which have shown similar efficacy at removing aldehydes, e.g., formaldehyde, from (meth)acrylic acid solutions, hydrazide compounds, such as carbohydrazide, is significantly benign from a health, safety and handling perspective.

When a hydrazide compound, such as carbohydrazide (CBZ), is allowed to come into contact with streams containing aldehydes and other carbonyl (non-acid) compounds, the carbonyls are consumed. For example, carbohydrazide appears to react preferentially with formaldehyde in solution with water, acetic acid, acrylic acid, and mixtures thereof.

Furthermore, by scavenging the aldehydes immediately downstream of the absorber (i.e., adding the hydrazide to the aqueous (meth)acrylic acid after its formation by absorption) which is the location of the highest concentration of formaldehyde, the method of the present invention may drastically improve the stability of the distillation columns, reducing fouling and allowing for increased asset utilization and operability. Surprisingly, it was also discovered that, contrary to previous reports concerning hydrazide scavenging of aldehydes, the products of carbohydrazide scavenging are soluble in the (meth)acrylic acid matrix. This obviates the need for either a heavy solvent or an organic sulfonic acid which was reported to greatly reduce deposits in U.S. Pat. No. 5,482,597.

After treatment with a hydrazide compound to remove at least a portion of the aldehyde compounds, the concentrated aqueous (meth)acrylic acid may then be subjected, in any suitable manner known to persons of ordinary skill in the relevant art, to further purification steps wherein at least some portions of the light and heavy end compounds are removed. For example, the concentrated aqueous (meth)acrylic acid may be purified by removing at least a portion of the one or more heavy end components, by any known method, such as for example, azeotropic or simple distillation. Furthermore, the concentrated aqueous (meth)acrylic acid may be purified by removing a portion of the one or more light end components, by any known method, such as for example, azeotropic or simple distillation.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

The following examples are illustrative of the invention but are not intended to limit its scope.

### EXAMPLES

### Example 1

A production unit sample of aqueous acrylic acid was aliquoted and the individual fractions charged with carbohydrazide. The samples were each heated to 60° C. for 30 min and individual aliquots analyzed for formaldehyde, benzaldehyde, furfural and maleic acid. The results are provided in Table 1 below.

TABLE 1

| | Wt/% | | | | | | |
|---|---|---|---|---|---|---|---|
| Carbohydrazide | 0.000 | 0.025 | 0.051 | 0.094 | 0.253 | 0.455 | 1.003 |
| Formaldehyde | 0.557 | 0.511 | 0.441 | 0.379 | 0.256 | 0.109 | 0.017 |
| Benzaldehyde | 0.018 | 0.018 | 0.018 | 0.017 | 0.017 | 0.017 | 0.003 |
| Furfural | 0.013 | 0.013 | 0.013 | 0.013 | 0.013 | 0.012 | 0.003 |
| Protoanemonin | 0.009 | 0.009 | 0.009 | 0.009 | 0.009 | 0.008 | 0.002 |
| Maleic acid | 0.305 | 0.325 | 0.319 | 0.308 | 0.314 | 0.317 | 0.309 |

### Example 2

A synthetic solution of aqueous acrylic acid was prepared by mixing flocculant grade acrylic acid (64.99 g), H₂O (35.01 g), formaldehyde (0.50 g, as 1.35 g of a 37% formalin solution), maleic acid (0.50 g) and propionaldehyde (0.50 g). An aliquot (17.41 g) which contained formaldehyde (2.89 mmol), maleic acid (0.79 mmol) and propionaldehyde (1.59 mmol) was removed and carbohydrazide (97% purity, 0.178 g, 1.91 mmol) added. The solution was mixed and heated for 30 min. at 49.5° C. An aliquot was removed and analyzed by ¹H NMR and compared to the original stock solution. No formaldehyde signals were detected by NMR and the bulk of the propionaldehyde was consumed. The propionaldehyde loss was based on the disappearance of the methyl and methylene group.

### Example 3

A solution of aqueous AA containing formaldehyde (0.557 wt. %, 0.468 eq. mol) was charged with carbohydrazide (0.768 eq. mol). The solution was kept at room temperature for 1 hour and analyzed by ¹H NMR. The sample was compared with an authentic sample and the formaldehyde and hydrate peaks at 5.4 and 4.95 ppm were found to be completely absent from the treated sample.

### Example 4

A sample of aqueous AA from a commercial production unit containing formaldehyde (0.557 wt. %, 12.33 eq. mol), furfural (0.013 wt. %, 0.09 eq. mol) and benzaldehyde (0.018 wt. %, 0.11 eq. mol) was charged with carbohydrazide (3.77 eq. mol). The sample was heated at 30° C. for 30 min. and allowed to sit overnight. The sample was subjected to a single stage flash on a rotory evaporator and afforded formaldehyde in the overheads (0.164 wt. %, 2 eq. mol)) and bottoms (0.021 wt. %, 0.41 eq. mol). A similar analysis for furfural and benzaldehyde was conducted in the overheads (0.005 wt %, 0.02 eq. mol, 0.007 wt. %, 0.02 eq. mol, respectively) and the bottoms (0.016 wt. %, 0.009 eq. mol, 0.034 wt. %, 0.02 eq. mol, respectively).

### Example 5

As a representative example, an aqueous acrylic acid solution comprising of acrylic acid (65 wt %), water (30 wt %), formaldehyde (0.65 wt %) was fed at a rate of 265 g/h to an azeotropic distillation column. The column is 33 mm in diameter and equipped with 30 Oldershaw trays. A steam heated reboiler loop was used to generate the vapor in the column. The feed was added to the middle section of the column, in this case tray 18. Methyl isobutylketone (MiBK) was added at the top as the reflux feed at a rate of 350 g/h. The overheads were condensed and allowed to phase separate and the organic layer returned as reflux. The aqueous layer was analyzed. The bottoms temperature was maintained via the steam controller and was set at 97-98° C. The column bottoms pressure was maintained at 200 mm Hg. A bottoms take-off in the reboiler loop afford the product. Hourly fractions were collected and analyzed for formaldehyde. The data in the table below show the values for the last hour of run time during a typical 5 h run (No additive).

In a separate experiment using the identical setup, an aqueous acrylic acid feed containing AA (65 wt %), water (30 wt %) and formaldehyde (0.65 wt %) was treated with carbohydrazide (0.29 mol). The mixture was stirred at room temperature for 16 hr and then fed to a azeotropic distillation column as described above. The results from the last hour are shown below in Table 2. Examination of the column during the distillation and after showed it to be free of any foulant or polymer.

TABLE 2

| | | | | Organic | | Aqueous | |
|---|---|---|---|---|---|---|---|
| | Feed | | Bottoms | Layer | | Layer | |
| No | .054 | mol | 0.00017 mol | 0.0020 | mol | 0.0463 | mol |
| additive | | | | | | | |
| With | 0.054 | mol | 3.32 × | 0.00046 | mol | 0.00344 | mol |
| Additive | | | 10−5 mol | | | | |

### Analytic Standards and Equipment

NMR data were obtained on a Varian Inova Instrument operating at 499.741 MHz. The one-dimensional ¹³C spectra were obtained at 120.46 MHz with a spectral width of 35000 Hz with a 2 second acquisition time and a 90° pulse of 11.1 microseconds. Gas chromatography was conducted using an Agilent HP 6890 with an FID detector. Formaldehyde determination was conducted on an HP 6890 using a packed column.

What is claimed is:

1. A method for reducing fouling of equipment during purification of (meth)acrylic acid in a process comprising:
A) producing a mixed product gas comprising (meth)acrylic acid, one or more aldehyde compounds, one or more light end compounds each having a lighter boiling point than (meth)acrylic acid, and one or more heavy end compounds each having a higher boiling point than (meth)acrylic acid;
B) producing aqueous (meth)acrylic acid from the mixed product gas comprising the (meth)acrylic acid, the one or more aldehyde compounds, the one or more light end compounds, the one or more heavy end compounds, and water;
C) removing at least a portion of the water from the aqueous (meth)acrylic acid to produce a concentrated aqueous (meth)acrylic acid;
D) purifying the concentrated aqueous (meth)acrylic acid by removing at least a portion of the one or more heavy end components; and
E) optionally, purifying the concentrated aqueous (meth)acrylic acid by removing an additional portion of the one or more light end components;
wherein the improvement comprises:
removing at least a portion of the one or more aldehyde compounds from the aqueous (meth)acrylic acid by adding at least one hydrazide:
1) during step B) of producing the aqueous (meth)acrylic acid; or
2) after step B), to the aqueous (meth)acrylic acid, and prior to any of the water removing and purifying steps C), D) and E); or
3) both 1) and 2),
wherein said hydrazide compound has the following formula:
H₂N—NHR₁
wherein R₁ is C(O)NH₂) or C(O)NHNH₂.

2. The method according to claim 1, wherein said hydrazide compound is a semicarbohydrazide.

3. The method according to claim 1, wherein said hydrazide compound is carbohydrazide.

4. The method according to claim 1, wherein said hydrazide compound is added in an amount of from 0.5 to 5 moles per 1 mole of aldehyde compound present in the aqueous (meth)acrylic acid.

5. The method according to claim 1, wherein step B), producing aqueous (meth)acrylic acid is accomplished by subjecting the mixed product gas to absorption with a solvent comprising water to remove at least a portion of the one or more light end compounds.

6. The method according to claim 1, wherein step D) is accomplished, at least in part, by distilling the concentrated aqueous (meth)acrylic acid.

7. The method according to claim 1, wherein optional step E) is accomplished, at least in part, by distilling the concentrated aqueous (meth)acrylic acid.

8. The method according to claim 1, wherein said (meth)acrylic acid is acrylic acid.

9. The method according to claim 1, wherein step A), producing a mixed product gas, is accomplished by vapor phase oxidation of an alkane, alkene, or mixtures thereof.
